(19) United States
(12) Patent Application Publication
(10) Pub. No.: US 2024/0006083 A1
(43) Pub. Date: Jan. 4, 2024

(54) SYSTEMS AND METHODS FOR AUTHORIZATION OF MEDICAL TREATMENTS USING AUTOMATED AND USER FEEDBACK PROCESSES

(71) Applicant: Aetna Inc., Hartford, CT (US)

(72) Inventors: Jonathan Singer, Hartford, CT (US); Isabel Vilar, Hartford, CT (US); Siva Koneru, Hartford, CT (US); Sherry Call, Hartford, CT (US); Chris Burnett, Hartford, CT (US); Celeste Burroughs, Hartford, CT (US); Theresa Wankum, Hartford, CT (US); Rinchen Lama, Hartford, CT (US); Srikanth Peruri, Hartford, CT (US)

(21) Appl. No.: 17/855,034

(22) Filed: Jun. 30, 2022

(51) Int. Cl. G16H 70/20; G16H 10/60; G16H 10/20

(52) U.S. Cl. CPC G16H 70/20; G16H 10/60; G16H 10/20

(57) ABSTRACT

In some instances, a method is provided. The method comprises: receiving, by a pre-certification authorization system (PCAS), treatment information indicating a medical treatment for a patient, wherein the treatment information is associated with a pre-certification request for the patient; determining, by the PCAS, whether to provide pre-certification approval for the medical treatment for the patient based on using a plurality of approval processors to determine at least two results, wherein the plurality of approval processors comprises a user feedback processor configured to generate a first result of the at least two results, and at least one autonomous processor configured to generate one or more second results of the at least two results, wherein the user feedback processor generates the first result based on user feedback from a user device.

### BACKGROUND

Patients and health care providers may seek authorization for medical treatments prior to performing the medical treatments. For instance, pre-certification (e.g., pre-authorization) is a process for obtaining approval to receive a particular medical service, treatment, and/or prescription medication. Given the numerous documents needed to be reviewed in order to provide an approval for the medical treatment, systems have been implemented to automate aspects of the pre-certification process, which helps expedite the overall process. For instance, by using a rules engine that comprises a plurality of rules, systems may be able to provide automation to aspects of the pre-certification process to obtain an approval for a patient faster. But, these automated pre-certification processes are not completely robust at this time for the numerous different types of medical procedures that require pre-certification nor are they able to take into account the complexity associated with each patient's individual circumstances and situations prior to providing approval. Accordingly, there exists a need for a technical solution to supplement the autonomous pre-certification approval systems in order to improve the automated pre-certification process.

### SUMMARY

In some examples, the present application may use automated processes as well as user feedback processes to provide pre-certification for a medical treatment for a patient. For instance, an enterprise system such as a pre-certification authorization system (PCAS) may obtain a request regarding pre-certification for performing a medical procedure for a patient. The PCAS may use multiple different processors configured to perform multiple pre-certification processes, and determine whether to provide approval for the medical procedure for the patient based on performing the different types of pre-certification processes. For example, one type of pre-certification process may be completely autonomous (e.g., by using a rules engine that comprises a plurality of rules and/or using predictive analytics/machine learning models). Further, another type of pre-certification process may prompt a user (e.g., the patient themselves or another individual associated with the patient such as an employee for a health care provider) to provide further feedback associated with the patient and/or the medical procedure. Each type of pre-certification process may provide a result, and the PCAS may determine whether to provide approval for the medical procedure for the patient based on the results from the different pre-certification processes and/or determine the duration period for an inpatient stay. Additionally, and/or alternatively, the PCAS may further be configured to determine whether to extend an initial treatment facility stay duration using machine learning models.

In one aspect, a method is provided. The method comprises: receiving, by a pre-certification authorization system (PCAS), treatment information indicating a medical treatment for a patient, wherein the treatment information is associated with a pre-certification request for the patient; determining, by the PCAS, whether to provide pre-certification approval for the medical treatment for the patient based on using a plurality of approval processors to determine at least two results, wherein the plurality of approval processors comprises a user feedback processor configured to generate a first result of the at least two results, and at least one autonomous processor configured to generate one or more second results, of the at least two results, wherein the user feedback processor generates the first result based on user feedback from a user device; and providing, by the PCAS and based on the at least two results from the plurality of approval processors, authorization information indicating whether the pre-certification request for the patient is approved.

In some instances, the treatment information indicates the medical treatment for the patient, identification information associated with the patient, a medical condition of the patient, and a medical provider providing the medical treatment.

In some examples, the at least one autonomous processor comprises a rules processor and a predictive processor, and wherein determining whether to provide the pre-certification approval for the medical treatment for the patient is based on using a hierarchy indicating an order to use the rules processor, the predictive processor, and the user feedback processor.

In some variations, determining whether to provide the pre-certification approval for the medical treatment for the patient comprises: determining, using the rules processor and based on the hierarchy, a third result based on applying one or more rules to the treatment information; providing, based on the hierarchy, the third result to the user feedback processor; in response to receiving the first result, providing, using the user feedback processor, questionnaire information associated with the treatment information to a user device; and determining, using the user feedback processor, the first result based on user feedback from the user device.

In some instances, determining whether to provide the pre-certification approval for the medical treatment for the patient further comprises: providing, based on the hierarchy, the first result to the predictive processor; determining, using the predictive processor and based one or more machine learning models, a fourth result; and providing the fourth result to a stay processor.

In some examples, determining whether to provide the pre-certification approval for the medical treatment for the patient further comprises: based on the fourth result indicating approval of the pre-certification request for the patient, generating the authorization information; and providing the authorization information indicating the approval of the pre-certification request to a health care treatment administration system.

In some variations, determining whether to provide the pre-certification approval for the medical treatment for the patient further comprises: based on the first result indicating approval of the pre-certification request for the patient, providing the first result to a stay processor; and providing the authorization information indicating the approval of the pre-certification request to a health care treatment administration system.

In some instances, determining whether to provide the pre-certification approval for the medical treatment for the patient further comprises: retrieving the questionnaire information from a decision repository based on the medical treatment indicated by the treatment information.

In some examples, the method further comprises: determining an approval of the pre-certification request based on the at least two results; in response to the approval, determining an approved treatment facility stay duration for the patient after the patient undergoes the medical treatment; and generating the authorization information, wherein the authorization information indicates the approval of the pre-certification request and the approved treatment facility stay duration for the patient.

In some instances, determining the approved treatment facility stay duration for the patient after the patient undergoes the medical treatment comprises: determining, using one or more parameters and the treatment information, an initial treatment facility stay duration, wherein each of the one or more parameter indicates a recommended stay duration associated with a particular type of medical treatment; and inputting the initial treatment facility stay duration into one or more machine learning models to determine an extended treatment facility stay duration, wherein the approved treatment facility stay duration is the extended treatment facility stay duration determined by the one or more machine learning models.

In another aspect, a pre-certification authorization system (PCAS) is provided. The PCAS comprises: an intake system configured to receive treatment information indicating a medical treatment for a patient, wherein the treatment information is associated with a pre-certification request for the patient; a user feedback processor configured to generate a first result based on user feedback from a user device; at least one autonomous processor configured to generate one or more second results; and a stay processor configured to: determine whether to provide pre-certification approval for the medical treatment for the medical treatment for the patient based on the first result and the one or more second results; and provide authorization information indicating whether the pre-certification request for the patient is approved.

In some examples, the treatment information indicates the medical treatment for the patient, identification information associated with the patient, a medical condition of the patient, and a medical provider providing the medical treatment.

In some variations, the at least one autonomous processor comprises a rules processor and a predictive processor.

In some instances, the rules processor is configured to: determine a third result based on applying one or more rules to the treatment information and a hierarchy indicating an order to use the rules processor, the predictive processor, and the user feedback processor; and provide, based on the hierarchy, the third result to the user feedback processor; and wherein the user feedback processor is configured to generate the first result based on the third result and the user feedback from the user device.

In some examples, the user feedback processor is further configured to: provide the first result to the predictive processor, and wherein the predictive processor is configured to: determine, based on one or more machine learning models, a fourth result; and provide the fourth result to the stay processor.

In some variations, the stay processor is configured to: based on the fourth result indicating approval of the pre-certification request for the patient, generate the authorization information, and wherein providing the authorization information comprises providing the authorization information indicating the approval of the pre-certification request to a health care treatment administration system.

In some instances, the user feedback processor is further configured to: based on the first result indicating approval of the pre-certification request for the patient, provide the first result to the stay processor, and wherein the stay processor is configured to: generate the authorization information based on receiving the first result, and wherein providing the authorization information comprises providing the authorization information indicating the approval of the pre-certification request to a health care treatment administration system.

In some examples, the stay processor is configured to: in response to determining that the pre-certification request for the patient has been approved, determine an approved treatment facility stay duration for the patient after the patient undergoes the medical treatment; and generate the authorization information, wherein the authorization information indicates the approval of the pre-certification request and the approved treatment facility stay duration for the patient.

In some variations, the stay processor is configured to determine the approved treatment facility stay duration for the patient after the patient undergoes the medical treatment by: determining, using one or more parameters and the treatment information, an initial treatment facility stay duration, wherein each of the one or more parameter indicates a recommended stay duration associated with a particular type of medical treatment; inputting the initial treatment facility stay duration into one or more machine learning models to determine an extended treatment facility stay duration; and determining the approved treatment facility stay duration as the extended treatment facility stay duration determined by the one or more machine learning models.

In a third aspect, a non-transitory computer-readable medium having processor-executable instructions stored thereon is provided. The processor-executable instructions, when executed, facilitate: receiving treatment information indicating a medical treatment for a patient, wherein the treatment information is associated with a pre-certification request for the patient; determining whether to provide pre-certification approval for the medical treatment for the patient based on using a plurality of approval processors to determine at least two results, wherein the plurality of approval processors comprises a user feedback processor configured to generate a first result of the at least two results, and at least one autonomous processor configured to generate one or more second results, of the at least two results, wherein the user feedback processor generates the first result based on user feedback from a user device; and providing, based on the at least two results from the plurality of approval processors, authorization information indicating whether the pre-certification request for the patient is approved.

All examples and features mentioned above may be combined in any technically possible way.

### BRIEF DESCRIPTION OF THE DRAWINGS

The present application will be described in even greater detail below based on the exemplary figures. The application is not limited to the examples described below. All features described and/or illustrated herein can be used alone or combined in different combinations in examples of the application. The features and advantages of various examples of the present application will become apparent by reading the following detailed description with reference to the attached drawings which illustrate the following:

FIG. 1 shows a simplified block diagram depicting an exemplary computing environment in accordance with one or more examples of the present application.

FIG. 2 shows a simplified block diagram of one or more systems within the exemplary environment of FIG. 1.

FIG. 3 shows another simplified block diagram depicting an exemplary pre-certification authorization system (PCAS) in accordance with one or more examples of the present application.

FIG. 4 shows an exemplary process for providing pre-certification approval for a medical treatment for a patient in accordance with one or more examples of the present application.

FIG. 5 shows another exemplary process for providing pre-certification approval for a medical treatment for a patient in accordance with one or more examples of the present application.

FIG. 6 shows a display screen displaying a user feedback form for the medical treatment for the patient in accordance with one or more examples of the present application.

FIG. 7 shows another display screen displaying a user feedback form for the medical treatment for the patient in accordance with one or more examples of the present application.

FIG. 8 shows yet another display screen displaying a user feedback form for the medical treatment for the patient in accordance with one or more examples of the present application.

### DETAILED DESCRIPTION

Examples of the presented application will now be described more fully hereinafter with reference to the accompanying FIGs., in which some, but not all, examples of the application are shown. Indeed, the application may be embodied in any different forms and should not be construed as limited to the examples set forth herein; rather, these examples are provided so that the disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on”.

Systems, methods, and computer program products are herein disclosed that provide pre-certification approval for a medical treatment for a patient using two or more pre-certification approval processes. Pre-certification may refer to an enterprise organization (e.g., a healthcare payer organization such as a healthcare insurance company) providing authorization for a healthcare service (e.g., procedure, test, surgery, and/or other types of medical treatments) or a healthcare product (e.g., a prescription or medication) before the healthcare service or product is provided. For example, the patient or a healthcare provider (e.g., a hospital or clinic) may provide a request to the enterprise organization for pre-certification for a medical treatment/medical procedure. The enterprise organization may perform a pre-certification process to determine whether to approve the medical treatment prior to the performance of the medical treatment (e.g., prior to performing the surgery).

Traditionally, systems were in place to perform an automated process for at least some aspects of pre-certification approval. For instance, the traditional systems may include a rules engine/processor that uses one or more set rules and/or a predictive engine/processor that uses predictive analytics/machine learning models to automate and approve the pre-certification request. For instance, using the rules engine and predictive analytics/machine learning models, traditional systems may determine whether to approve the scheduled medical treatment for the patient. Furthermore, other types of traditional systems may obtain user feedback (e.g., by providing questionnaires to a user) during the pre-certification process. Based on the responses obtained from the user, these traditional systems may determine whether to approve the medical treatment for the patient. However, these traditional systems have several limitations. For instance, when determining whether to approve the pre-certification request, the automated process/engine (e.g., using the rules and/or predictive analytics/machine learning models to determine whether to approve the medical treatment for the patient) might not be able to take into account the patient's particular situation or the circumstances that led to the pre-certification request being submitted. For instance, the rules engine may include rules that are based on previous medical treatments performed by a medical provider, and determining to approve based on the enterprise organization's experience with working with the particular medical provider. The predictive analytics/machine learning models may be based on (e.g., trained using) demographic data associated with a population of patients. As such, a traditional automated processor/engine might not take into account the patient's unique situation when determining whether to approve the pre-certification request for the patient. Similarly, traditional systems that utilize user feedback (e.g., by providing a questionnaire to a user) may be static in nature and not able to take into account certain parameters that are outside of their purview, which also may overlook granting pre-certification approvals for certain types of medical treatments for certain patients. Given that these pre-certifications are important, especially to ease a patient's mind when making a decision as to whether to undergo such medical treatments, the present application uses multiple different types of pre-certification approval processes in order to provide expedited pre-certification approval for medical treatments for patients.

Additionally, and/or alternatively, whereas the traditional systems (e.g., traditional automated systems that used rules and predictive analytics or traditional systems that solely used user feedback/questionnaires) determined whether to approve the pre-certification request for the patient, the present application further provides additional information after the pre-certification request has been approved. For instance, the present application may also determine whether to extend an approved duration of stay (e.g., hospital or treatment facility stay) for the patient after the patient undergoes the medical treatment. For instance, based on the medical procedure and/or other factors indicated by the pre-certification request, the present application may first determine an approved treatment facility stay duration (e.g., 3 days) after the medical treatment (e.g., the surgery) was performed, and subsequently, use predictive analytics and/or machine learning models to determine whether to extend the approved hospital or treatment facility stay (e.g., extend the approved hospital stay from 3 days to 5 days). This will be explained in further detail below.

Among other benefits, by using a dual pre-certification approval process, the present application may provide pre-certification approval at a significantly faster pace, which may greatly reduce patients' anxiety as to whether their medical treatment has been pre-approved. Furthermore, by automating the process, the present application may better standardize the pre-certification approval process, and this may further provide better consistency. In addition, when new internal or external initiatives are initiated, the present application may be able to expedite the inclusion of the new initiatives as the process becomes more automated and/or streamlined.

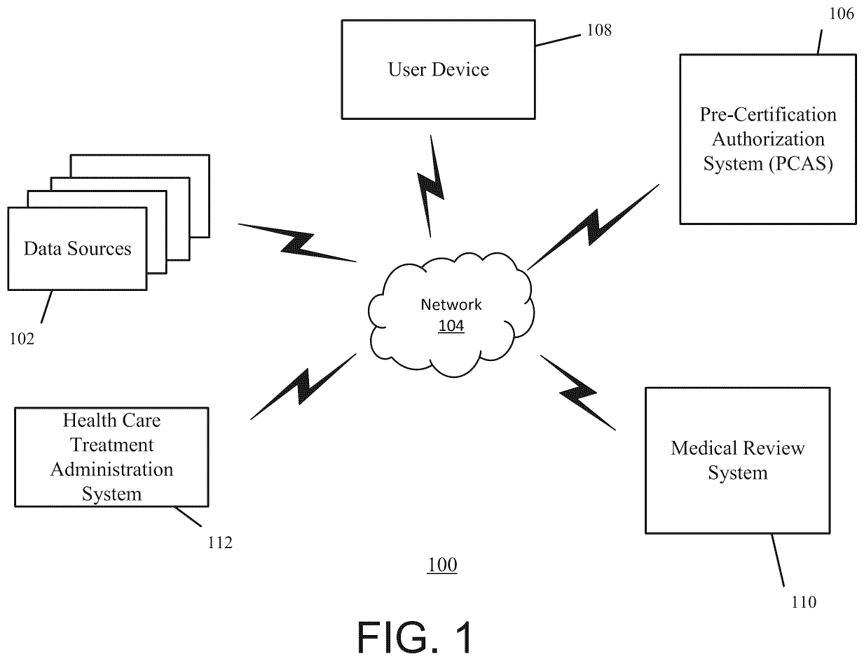

FIG. 1 is a simplified block diagram depicting an exemplary environment 100 in accordance with an example the present application. The environment 100 includes data sources 102, a network 104, a pre-certification authorization system (PCAS) 106, a medical review system 110, a user device 108, and a health care treatment administration system 112. As used herein, the systems and devices within the environment 100 include one or more devices, servers, network elements, and/or other types of computing devices.

The systems within the environment 100 may be operatively coupled (e.g., in communication with) other systems within the environment 100 via the network 104. The network 104 may be a global area network (GAN) such as the Internet, a wide area network (WAN), a local area network (LAN), or any other type of network or combination of networks. The network 104 may provide a wireline, wireless, or a combination of wireline and wireless communication between the systems and/or other components within the environment 100.

The data sources 102 include one or more devices, computing devices, systems, and/or other entities that provide data (e.g., pre-certification requests) to the PCAS 106. For example, for each pre-certification request, a patient and their medical provider (e.g., hospital or clinic) may provide information associated with the patient and/or medical treatment in order to obtain pre-certification approval with an enterprise organization (e.g., a healthcare payer organization). The data sources 102 may include a computing device that is configured to generate the pre-certification request and provide the pre-certification request to the PCAS 106 (e.g., an intake channel for enterprise organization). For instance, the computing device may access a provider portal, which displays one or more display screens onto the computing device. Using the display screens, the computing device may obtain pre-certification request information (e.g., patient name, medical treatment required, and other information associated with the patient and/or medical treatment). Subsequently, the computing device provides the pre-certification request information to the PCAS 106 so that the PCAS 106 may process the pre-certification request.

Additionally, and/or alternatively, the pre-certification requests may be provided to the PCAS 106 by other data sources 102. For instance, the pre-certification requests may be provided via a phone call, fax, or using electronic data interchange (EDI). As such, the data sources 102 may include and/or be associated with any type of devices configured to obtain the pre-certification request information and provide the pre-certification request information to the PCAS 106. For instance, the patient and/or the medical provider may provide the pre-certification request information via telephone. An operator, using a computing device, may input the pre-certification request information based on the phone call, and provide the pre-certification request information to the PCAS 106.

The PCAS 106 includes one or more processors, computing devices, and/or computing systems that are configured to determine whether to approve the pre-certification request for the medical treatment for the patient. For instance, the PCAS 106 may obtain the pre-certification request information from the data sources 102 and perform a plurality of pre-certification request processes to determine whether to provide pre-certification approval for the medical treatment for the patient. For instance, as will be described in further detail below, the PCAS 106 may perform an autonomous process (e.g., using a rules engine/processor and/or a predictive engine/processor) as well as perform a feedback process (e.g., using feedback processor/engine) to determine whether to approve the pre-certification request. Based on the determination, the PCAS 106 may provide authorization information to the medical review system 110 and/or the health care treatment administration system 112. Additionally, and/or alternatively, the PCAS 106 may determine an approved treatment facility stay duration for the patient. For instance, after the medical treatment (e.g., the surgery) has been performed, the patient may be required to stay in the hospital for a certain length of time. Accordingly, after approving the pre-certification request, the PCAS 106 may also determine, using one or more rules (e.g., parameters), a treatment facility stay duration for the patient after the patient undergoes the medical treatment. For instance, the PCAS 106 may determine a treatment facility stay duration of 3 days. Additionally, and/or alternatively, the PCAS 106 may use predictive analytics and/or machine learning models to determine whether to extend the determined treatment facility stay duration. For instance, using one or more machine learning models, the PCAS 106 may determine to extend the hospital stay of the patient from 3 days to 5 days.

The PCAS 106 is a computing system that is associated with the enterprise organization. The enterprise organization may be any type of corporation, company, organization, and/or other institution (e.g., a healthcare institution). The PCAS 106 may be implemented using one or more computing platforms, devices, servers, and/or apparatuses. In some variations, the PCAS 106 may be implemented as engines, software functions, and/or applications. In other words, the functionalities of the PCAS 106 may be implemented as software instructions stored in storage (e.g., memory) and executed by one or more processors.

Furthermore, for the feedback process, the PCAS 106 may be in communication with the user device 108. For instance, the PCAS 106 may provide information to the user device 108 and the user device may display the information to a user. The user may be the patient or an employee or other member associated with the healthcare provider. The user device 108 may obtain user input associated with responses for the patient, and provide the responses back to the PCAS 106. For example, the PCAS 106 may provide one or more questions (e.g., a questionnaire) associated with the patient (e.g., the patient history) and/or the medical treatment (e.g., the medical procedure to be performed on the patient). The user device 108 may display the questionnaire and provide the user input indicating responses or answers to the questionnaire back to the PCAS 106.

The user device 108 may be and/or include, but is not limited to, a desktop, laptop, tablet, mobile device (e.g., smartphone device, or other mobile device), smart watch, an internet of things (IOT) device, or any other type of computing device that generally comprises one or more communication components, one or more processing components, and one or more memory components. The user device 108 may be able to execute software applications managed by, in communication with, and/or otherwise associated with the enterprise organization. In some instances, the user device 108 may be a device associated with and/or included within the data sources 102. For instance, the user device 108 may be the same device that provided the original pre-certification request to the PCAS 106.

After the PCAS 106 determines authorization information (e.g., information indicating whether the pre-certification request is approved), the PCAS 106 provides the authorization information to the health care treatment system 112 and/or the medical review system 110.

The medical review system 110 may be a system for an operator to provide further review regarding the pre-certification request. For instance, the PCAS 106 may determine that the authorization information is “pend” or pending for the pre-certification request. The medical review system 110 may display information for the pre-certification request, including information associated with the patient and/or the medical treatment. The operator, using the medical review system 110, may provide feedback such as approval of the pre-certification request. Then, the medical review system 110 may provide the feedback to the health care treatment administration system 112.

The medical review system 110 may be implemented using one or more computing platforms, devices, and/or apparatuses such as laptops, desktops, tablets, and so on. In some variations, the medical review system 110 may be implemented as engines, software functions, and/or applications. In other words, the functionalities of the medical review system 110 may be implemented as software instructions stored in storage (e.g., memory) and executed by one or more processors connected to a display device.

The health care treatment administration system 112 may be a system associated with a healthcare provider (e.g., a hospital or clinic). The health care treatment administration system 112 may receive information from the PCAS 106 and/or the medical review system 110. For instance, the health care treatment administration system 112 may receive information indicating the approval of the pre-certification request and/or a length of treatment facility stay (e.g., hospital stay) approved for the medical treatment (e.g., a 5 day approval for hospital stay after the medical treatment). In other words, the health care treatment administration system 112 may receive and display information indicating that the medical treatment for the patient has been pre-certified. In some examples, the health care treatment administration system 112 may receive the authorization information (e.g., the pre-certification approval of the medical treatment for the patient) directly from the PCAS 106. For instance, the PCAS 106, using the pre-certification processes (the autonomous process and the feedback process), may directly provide pre-certification approval. In other examples, the PCAS 106 may provide authorization information to the medical review system 110, and the medical review system 110 may provide pre-certification approval to the health care treatment administration system 112.

In some instances, the health care treatment administration system 112 may be a device associated with and/or included within the data sources 102. For instance, the health care treatment administration system 112 may be the same device that provided the original pre-certification request to the PCAS 106.

It will be appreciated that the exemplary system depicted in FIG. 1 is merely an example, and that the principles discussed herein may also be applicable to other situations—for example, including other types of devices, systems, and network configurations.

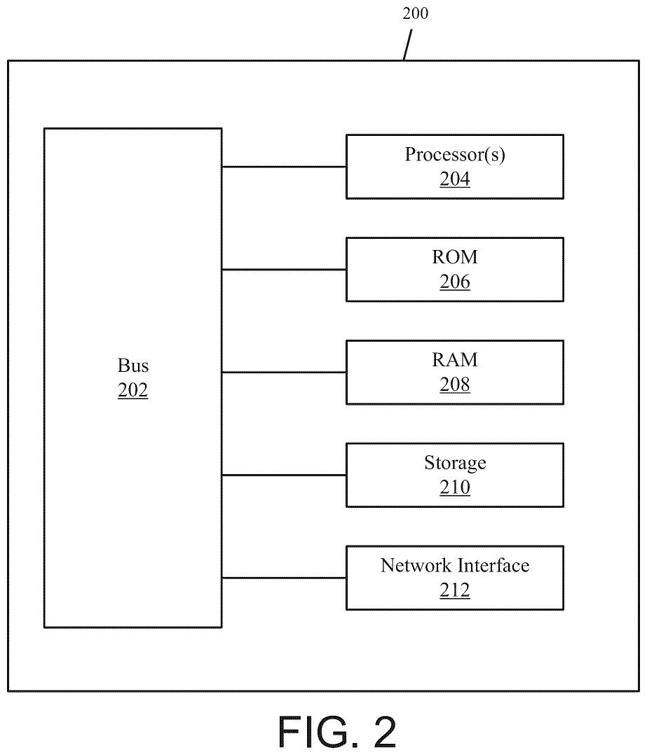

FIG. 2 is block diagram of an exemplary system or device within the environment 100. The system 200 includes a processor 204, such as a central processing unit (CPU), controller, unit, and/or logic, that executes computer executable instructions for performing the functions, processes, and/or methods described herein. In some examples, the computer executable instructions are locally stored and accessed from a non-transitory computer readable medium, such as storage 210, which may be a hard drive or flash drive. Read Only Memory (ROM) 206 includes computer executable instructions for initializing the processor 204, while the random-access memory (RAM) 208 is the main memory for loading and processing instructions executed by the processor 204. The network interface 212 may connect to a wired network or cellular network and to a local area network or wide area network, such as the network 104. The system 200 may also include a bus 202 that connects the processor 204, ROM 206, RAM 208, storage 210, and/or the network interface 212. The components within the system 200 may use the bus 202 to communicate with each other.

The system 200 of FIG. 2 may be used to implement the methods and systems described herein. For example, as will be explained below, the PCAS 106 may include the components of the system 200 and/or other components such as additional processors, engines, and/or systems.

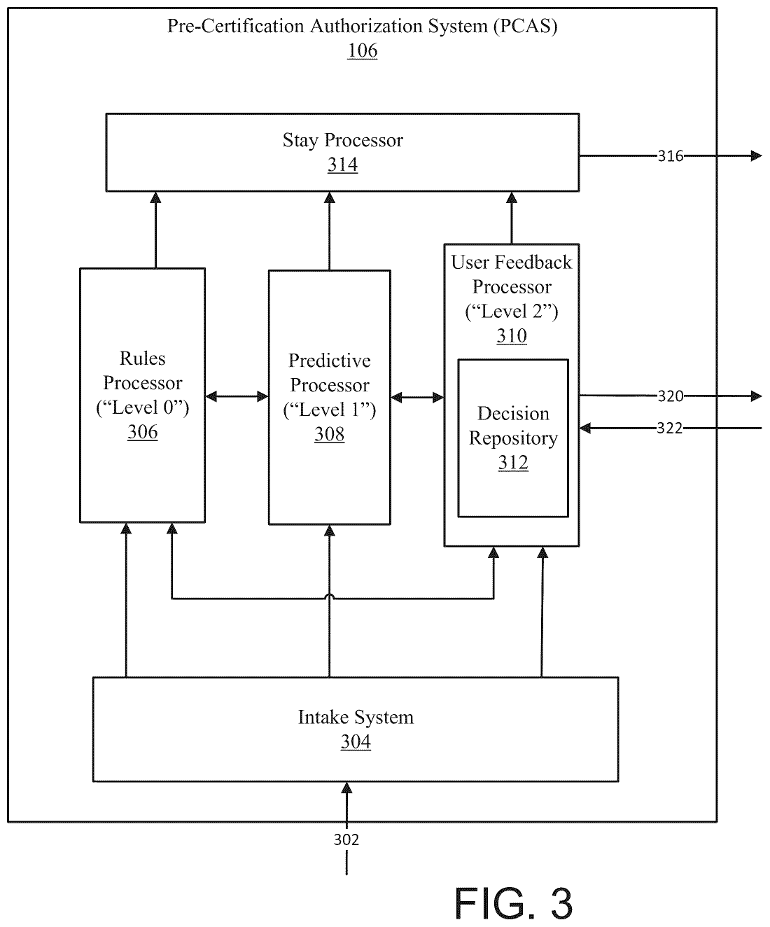

FIG. 3 is a block diagram of an exemplary PCAS 106 in accordance with one or more examples of the present application. The PCAS 106 includes an intake system 304, a rules processor 306 (“Level 0”), a predictive processor 308 (“Level 1”), a user feedback processor 310 (“Level 2”), and a stay processor 314.

While the system and processors 304, 306, 308, 310, and 314 are shown as separate processors, in some examples, one or more of the system/processors may be combined together and/or the functionalities of the system/processors may be implemented by a combined processor and/or computing device. Additionally, and/or alternatively, one or more of the system and processors 304, 306, 308, 310, and 314 may be separated into one or more additional processors (e.g., the stay processor 314 may include a first processor/engine for determining an initial duration of stay for the patient and a second processor/engine for determine whether to extend the initial duration of stay for the patient using machine learning models and/or predictive analytics). In some variations, the system and processors 304, 306, 308, 310, and 314 may be implemented as engines, software functions, and/or applications. In other words, the functionalities of the system and processors 304, 306, 308, 310, and 314, which are described below, might not be separate physical processors such as CPUs, and may be implemented as software instructions stored in a storage (e.g., memory) and executed by one or more processors, such as the storage 210 and processor(s) 204 in FIG. 2.

The user feedback processor 310 includes a decision repository 312. In some instances, the decision repository 312 may be separate from the user feedback processor 310. The decision repository 312 may be any type of storage medium, location, and/or memory that is capable of storing information. The decision repository 312 may be and/or include a computer-usable or computer-readable medium such as, but not limited to, an electronic, magnetic, optical, electromagnetic, infrared, or semiconductor computer-readable medium. More specific examples (e.g., a non-exhaustive list) of the computer-readable medium may include the following: an electrical connection having one or more wires; a tangible medium such as a portable computer diskette, a hard disk, a time-dependent access memory (RAM such as the RAM 208), a ROM such as ROM 206, an erasable programmable read-only memory (EPROM or Flash memory), a compact disc read-only memory (CD ROM), or other tangible optical or magnetic storage device.

In operation, the intake system 304 may receive treatment information 302 from a data source 102. For example, the data source 102 may provide the treatment information 302 indicating a pre-certification request for a medical treatment for a patient. The treatment information 302 may include, but is not limited to, the identification of a specific medical treatment or procedure for the patient, a medical condition of the patient, the medical provider providing the medical treatment, identification information for the patient, and/or the facility where the medical treatment will be performed. The medical treatment or procedure for the patient may include, but is not limited to, cataract surgery, hip & knee arthroplasty, shoulder arthroplasty & video electroencephalogram (EEG), endoscopic nasal balloon dilation, hip surgery to repair impingement syndrome, function E. sinus surgery, spinal fusion scoliosis, venous ligation, artificial disc, breast reconstruction, gastroplasty, and/or orthognathic surgery.

After receiving the treatment information 302, the intake system 304 may process the treatment information and route the treatment information 302 to one or more processors such as the rules processor 306, the predictive processor 308, and/or the user feedback processor 310. The rules processor 306, the predictive processor 308, and/or the user feedback processor 310 may be hierarchically orchestrated. The intake system 304 may route the treatment information 302 based on the hierarchy of these processors 306, 308, and 310 and/or the treatment information 302.

For instance, the intake system 304 may distribute the treatment information 302 to the processors based on a hierarchy for the processors. For instance, the intake system 304 may provide the treatment information 302 to a first processor (e.g., the rules processor 306) and further instructions for the first processor to forward their result to a second processor (e.g., the user feedback processor 310). Additionally, and/or alternatively, the intake system 304 may provide instructions for the second processor to forward their result to a third processor (e.g., the predictive processor 308), and the third processor may forward their result to the stay processor 314. In some examples, based on one or more of the processors indicating the result to be approved, the processor may provide the result to the stay processor 314. For instance, based on the user feedback processor 310 indicating approval, the user feedback processor 310 may provide the result directly to the stay processor 314, and might not provide the result to the next processor in the hierarchy (e.g., the predictive processor 308).

In some instances, the hierarchy may be based on the treatment information 302 for the patient (e.g., based on the type of the medical treatment indicated by the treatment information 302 and/or the medical provider indicated by the treatment information 302). For instance, for a first type of medical treatment (e.g., hip & knee arthroplasty), the intake system 304 may determine the hierarchy of processors associated with the medical treatment (e.g., from the rules processor 306 to the predictive processor 308 to the user feedback processor 310). The intake system 304 may then provide the treatment information 302 to the processors based on the determined hierarchy. For a second type of medical treatment (e.g., artificial disc surgery), the intake system 304 may determine the hierarchy of processors associated with the second type of medical treatment (e.g., from the user feedback processor 312 to the predictive processor 308). The intake system 304 may then provide the treatment information 302 to the processors based on the determined hierarchy.

The rules processor 306 (e.g., a rules engine) may include a plurality of rules for one or more of the medical treatments. For instance, the plurality of rules may include and/or indicate defined exemptions associated with the medical treatments. The rules processor 306 may compare the rules with the treatment information 302 to determine whether to provide pre-certification approval for the medical treatment for the patient. Afterwards, the rules processor 306 may determine a result (e.g., approval of the medical treatment for the patient or pending), and provide the result to another processor (e.g., the predictive processor 308, the stay processor 314, and/or the user feedback processor 310). In some instances, the rules may indicate particular medical providers (e.g., hospitals, clinics, medical personnel such as doctors, and/or other types of medical providers) that may automatically receive approval. For instance, the enterprise organization may have previous experience(s) with one or more medical providers. The previous experience(s) may be based on previous pre-certification approvals for other patients. A rule within the rules processor 306 may indicate to automatically approve the medical provider based on the previous experience(s). As such, the rules processor 306 may compare the rules with the treatment information 302 (e.g., the medical provider that provided the pre-certification request), and determine a result based on the comparison. In other words, the rules processor 306 may approve the pre-certification request based on previous experience(s) with the medical providers.

The predictive processor 308 (e.g., a predictive engine) may include and/or use predictive analytics and/or machine learning models for one or more of the medical treatments. For instance, the predictive processor 308 may input information from the treatment information 302 into the predictive analytics and/or machine learning models to determine whether to provide pre-certification approval for the medical treatment for the patient. Afterwards, the predictive processor 308 may determine a result (e.g., approval of the medical treatment for the patient or pending), and provide the result to another processor (e.g., the rules processor 306, the user feedback processor 310, and/or stay processor 314). In some instances, the predictive processor 308 may train the machine learning models using demographic data associated with a population of patients and their pre-certification requests and/or approvals. Additionally, and/or alternatively, the predictive processor 308 may use non-clinical historical data associated with the population of patients to train the machine learning models.

The user feedback processor 310 may use user feedback to determine whether to provide pre-certification approval for the medical treatment for the patient, and provide the determination to another processor (e.g., the rules processor 306, the predictive processor 308, and/or the stay processor 314). For instance, the user feedback processor 310 may provide questionnaire information 320 associated with the medical treatment to a user device such as user device 108. A user (e.g., the patient and/or a person associated with a health care provider) may provide user feedback 322 to the questionnaire information. The user feedback may indicate responses or answers to the questionnaire information. The user feedback processor 310 may receive the user feedback 322 to the questionnaire information 320 and use the user feedback to determine whether to provide pre-certification approval for the medical treatment for the patient.

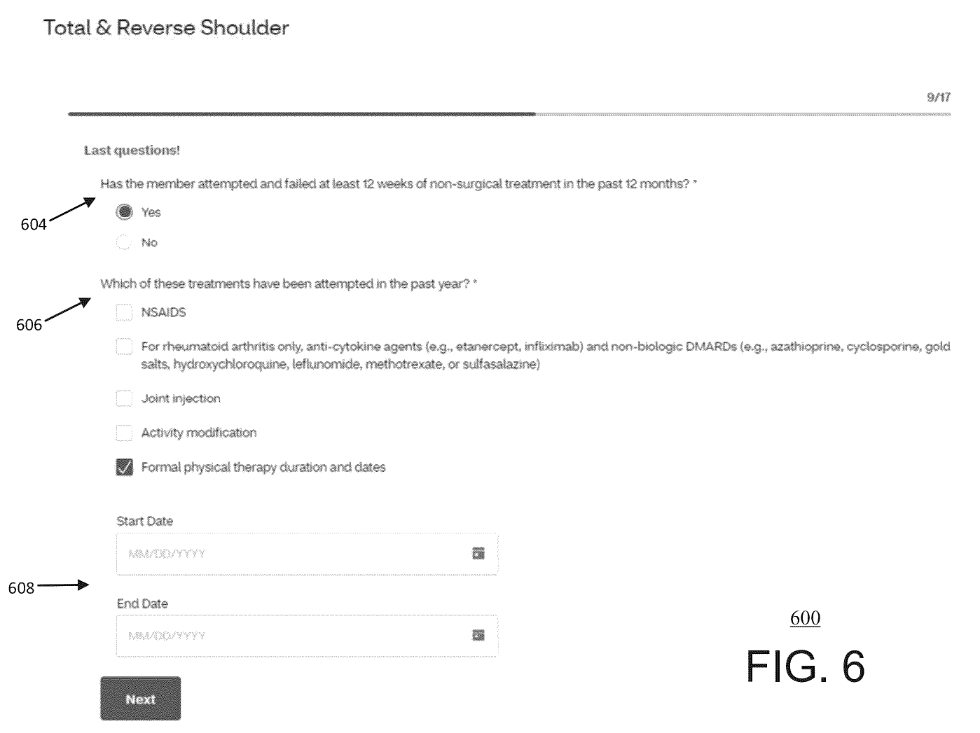

FIG. 6 shows a display screen 600 displaying a user feedback form for the medical treatment for the patient in accordance with one or more examples of the present application. For instance, the user feedback processor 310 may provide questionnaire information indicating one or more questions for the user. The user device 108 may display the questions such as questions 604, 606, and 608 for the surgery (e.g., the total & reverse shoulder surgery). Using the user device 108, the user may provide user input indicating responses to the questions 604, 606, and 608. In some instances, each response to the question may prompt one or more additional questions. For instance, based on the response to question 604, question 606 may show up on the display screen 600. Based on the answer to question 606, question 608 may show up on the display screen 600. As such, by the questionnaire information indicating a tiered question set (e.g., responses to one question such as question 604 leads to further questions such as questions 606 and/or 608), the user feedback processor 310 may be in a better position for determining whether to provide the pre-certification approval for the medical treatment for the patient as the questions/answers become more specific.

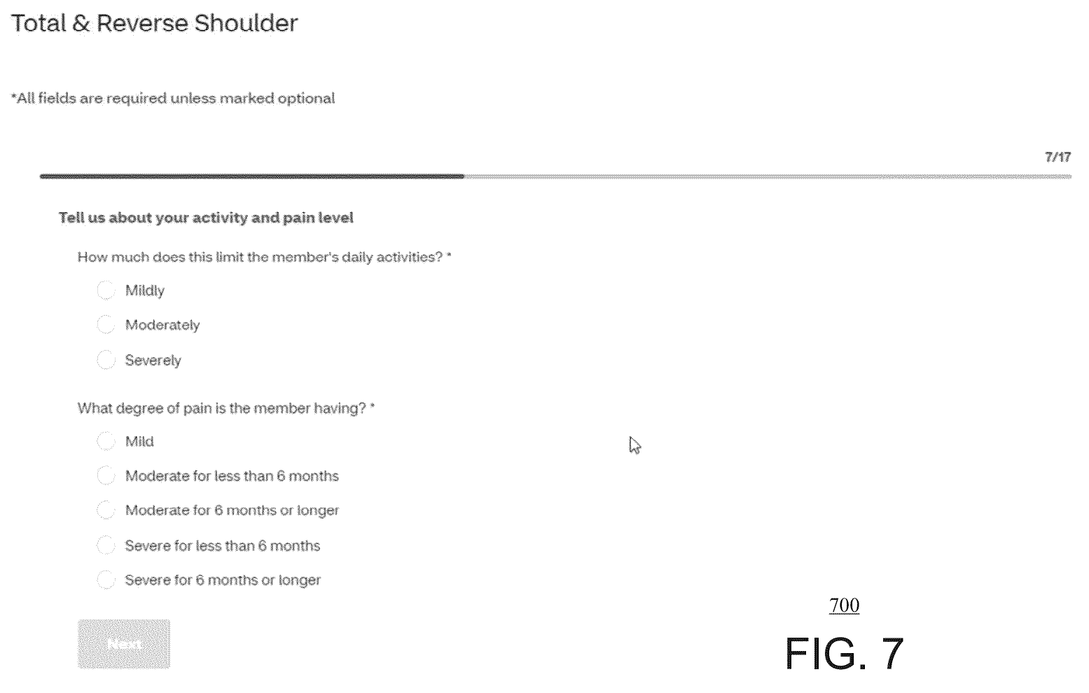

FIG. 7 shows another display screen 700 displaying a user feedback form for the medical treatment for the patient in accordance with one or more examples of the present application. For instance, similar to display screen 600, display screen 700 shows questions for the total & reverse shoulder surgery.

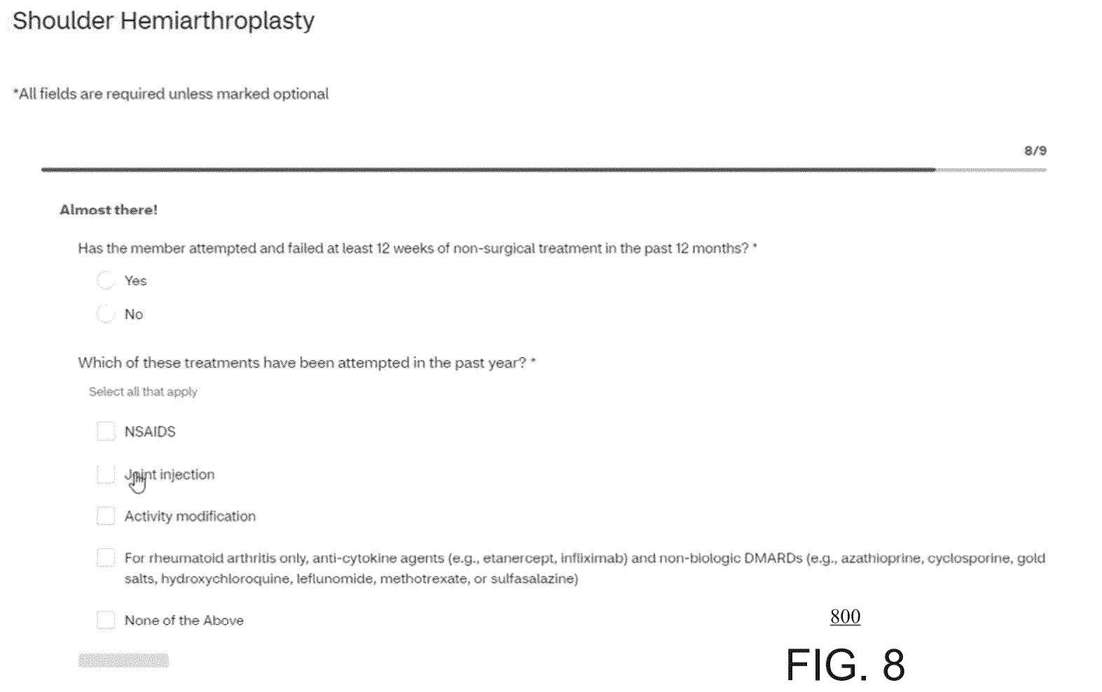

In some instances, the questionnaire information may be specific to a particular medical treatment and/or patient. For instance, the decision repository 312 may store questionnaire information (e.g., a plurality of sets of questions) associated with a plurality of different medical treatments. The user feedback processor 310 may retrieve the set of questions based on the treatment information 302. For instance, for a first treatment, the user feedback processor 310 may retrieve a first set of questions. For a second treatment, the user feedback processor 310 may retrieve a second set of questions. FIG. 8 shows yet another display screen 800 displaying a user feedback form for the medical treatment for the patient in accordance with one or more examples of the present application. For instance, similar to display screens 600 and 700, display screen 800 shows questions for a medical procedure. However, the medical procedure is for shoulder hemiarthroplasty, which may include a second set of questions, and one or more questions from the second set may be different from one or more questions from the first set of questions associated with the first medical treatment (e.g., the total & reverse shoulder surgery).

A computing device (e.g., the medical review system 110 and/or another computing device) may generate the questions for the different medical treatments and store the questions in the decision repository 312. For instance, individuals (e.g., medical personnel and/or others) from the enterprise organization may review guidelines (e.g., federal guidelines) and/or medical policies. Based on the review, the individuals may determine the questionnaire questions for the medical treatment and/or the structure of the questionnaire. The individuals may provide input to the PCAS 106 indicating the questions, and the PCAS 106 may store the questions and/or the structure of the questions within the decision repository 312.

The stay processor 314 may receive one or more results from the processors 306, 308, and/or 310. For instance, based on the hierarchy of the processors, the rules processor 306 may provide a result to the predictive processor 308, which may provide another result to the user feedback processor 310. The user feedback processor 310 may provide a result to the stay processor 314. The stay processor 314 may determine to provide authorization information indicating whether the medical treatment for the patient is approved to one or more devices based on the result. For instance, the stay processor 314 may be a routing processor that is configured to route the result to another entity within environment 100. For instance, the authorization information may indicate the medical treatment for the patient is approved. The stay processor 314 may provide the authorization information 316 indicating approval of the pre-certification request for the medical treatment for the patient to the health care treatment administration system 112. As mentioned previously, the health care treatment administration system 112 may be a system associated with a healthcare provider such as a hospital or clinic. The health care treatment administration system 112 may display information indicating approval of the pre-certification request, and may inform the patient regarding the approval accordingly.

In some instances, the authorization information 316 may indicate “pend” or pending, which indicates that the pre-certification request is to be reviewed further by another administrator. Based on the result indicating “pend”, the stay processor 314 may provide the authorization information 316 to the medical review system 110. The medical review system 110 may display information associated with the authorization information 316 and/or additional information associated with the treatment information 302. An administrator may review the displayed information and may provide user input indicating a decision (e.g., approval of the pre-certification request). Subsequently, the medical review system 110 may provide the decision or result to the health care treatment administration system 112 (e.g., the approval of the pre-certification request for the medical treatment for the patient).

Additionally, and/or alternatively, the stay processor 314 may determine a treatment facility stay duration approved for the medical treatment for the patient. For instance, after the medical treatment such as a surgery, the patient may be required to stay at the hospital for an extended amount of time to recover from the medical treatment. As such, the stay processor 314 may determine an approval for a treatment facility stay duration for the patient after the patient undergoes the medical treatment (e.g., a 3 day stay). In some instances, the stay processor 314 may use one or more parameters (e.g., rules) to determine the treatment facility stay duration for the patient. For instance, the stay processor 314 may store a plurality of parameters and the parameters may indicate a stay duration associated with the particular type of medical treatment. As such, the stay processor 314 may compare the plurality of parameters with the particular type of medical treatment indicated by the treatment information 302. Based on the comparison, the stay processor 314 may determine the approved treatment facility stay duration for the medical treatment for the patient. The stay processor 314 may provide information 316 (e.g., the authorization information) to another entity such as the health care treatment administration system 112. The authorization information 316 may indicate that the pre-certification request has been approved and may further indicate an approved treatment facility stay duration for the patient after the medical treatment is performed.

Additionally, and/or alternatively, the stay processor 314 may use one or more predictive analytics and/or machine learning models (e.g., artificial intelligence models) to determine whether to extend the approved treatment facility stay duration for the medical treatment for the patient. For instance, based on the comparison of the plurality of parameters with the treatment information 302, the stay processor 314 may determine an initial approved treatment facility stay duration (e.g., 3 days). Then, the stay processor 314 may use one or more machine learning models to determine whether to extend the approved treatment facility stay duration. For instance, based on using the machine learning models, the stay processor 314 may determine to extend the approved treatment facility stay duration to an extended treatment facility stay duration (e.g., 5 days). The stay processor 314 may input the treatment information 302 and/or the initial approved treatment facility stay duration into the machine learning model, and the machine learning model may output the extended stay duration. After determining the extended stay duration, the stay processor 314 may provide the authorization information 316 to the health care treatment administration system 112. The authorization information 316 may indicate that the pre-certification request has been approved and may further indicate an approved extended treatment facility stay duration for the patient after the medical treatment is performed.

It will be appreciated that the exemplary system depicted in FIG. 3 is merely an example, and that the principles discussed herein may also be applicable to other situations—for example, including other types of devices, processors, engines, and/or systems. For instance, as explained above, the functionalities of the system and processors 304, 306, 308, 310, and 314 may be implemented by software instructions, one or more combined processors, and/or one or more computing devices.

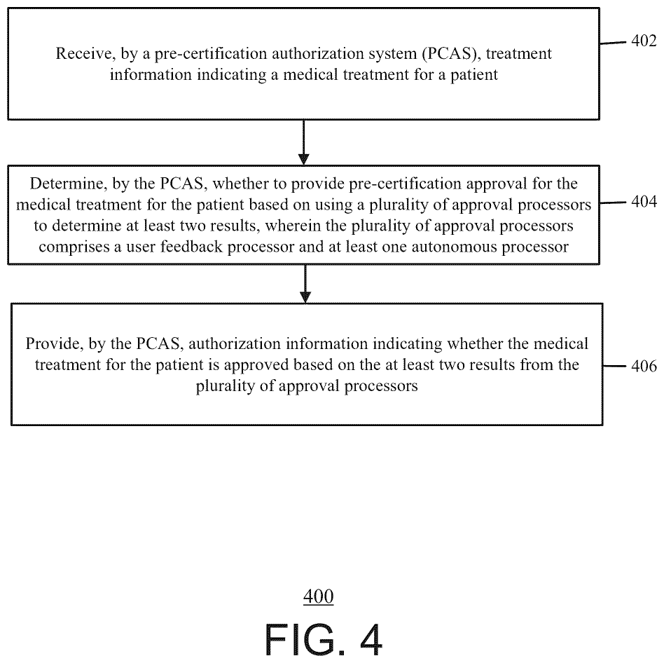

FIG. 4 shows an exemplary process for providing pre-certification approval for a medical treatment for a patient in accordance with one or more examples of the present application. The process 400 may be performed by the environment 100 and the exemplary PCAS 106 shown in FIG. 3; however, it will be recognized that any suitable environment and system may be used and that any of the following blocks may be performed in any suitable order.

In operation, at block 402, the PCAS 106 receives treatment information indicating a medical treatment for a patient. For instance, the treatment information may be associated with a request for pre-certification of the medical treatment for the patient. At block 404, the PCAS 106 determines whether to provide pre-certification approval for the medical treatment for the patient based on using a plurality of approval processors to determine at least two results. The plurality of approval processors comprises a user feedback processor (e.g., the user feedback processor 310) and at least one autonomous processor (e.g., the rules processor 306 and/or the predictive processor 308). For instance, the rules processor 306 and/or the predictive processor 308 may provide a first result (e.g., “pend”) and the user feedback processor 310 may provide a second result (e.g., “approve”). The stay processor 314 may determine a final result (e.g., “approve”) based on the first and second results from the processors 306, 308, and/or 310. For example, based on the hierarchy of the processors, the rules processor 306 may provide their result to the user feedback processor 310. The user feedback processor 310 may provide their result to the predictive processor 308, and the predictive processor 308 may provide the result (e.g., “approve”) to the stay processor 314. At block 406, the PCAS 106 provides authorization information (e.g., “approve”) indicating whether the medical treatment for the patient is approved based on the at least two results from the plurality of approval processors. For instance, based on the final result being approve, the PCAS 106 may provide the authorization information to the health care treatment administration system 112. Additionally, and/or alternatively, the PCAS 106 may determine an approved treatment facility stay duration for the patient after performance of the medical treatment. For instance, the PCAS 106 may use one or more parameters and/or predictive analytics/machine learning models to determine an approved treatment facility stay duration and/or an extended treatment facility stay duration. The PCAS 106 may provide the approved treatment facility stay duration to the health care treatment administration system 112.

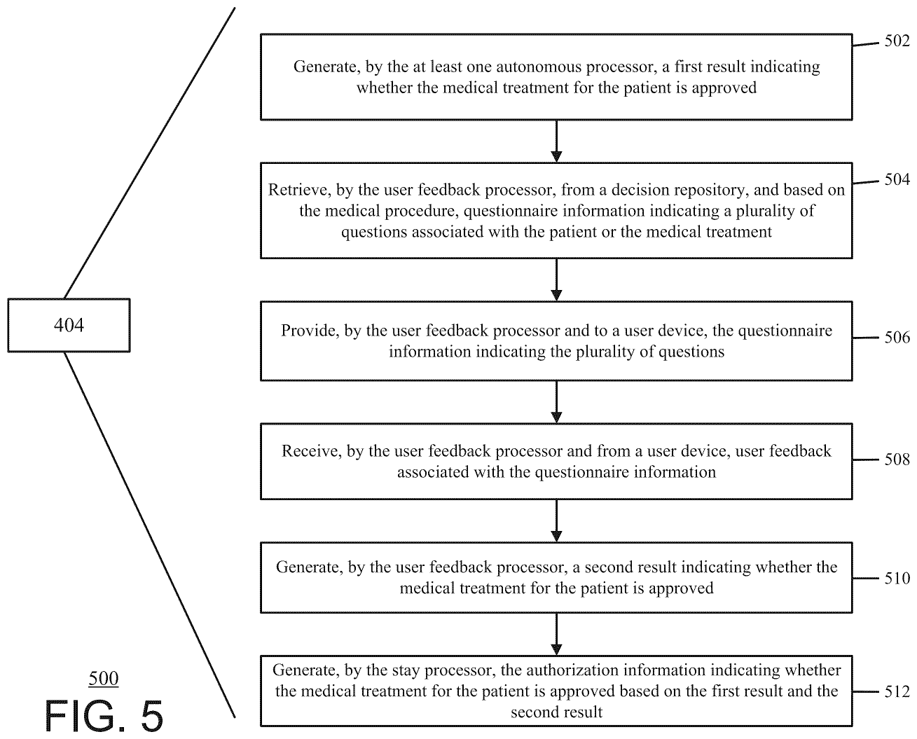

FIG. 5 shows another exemplary process for providing pre-certification approval for a medical treatment for a patient in accordance with one or more examples of the present application. The process 500 may be performed by the environment 100 and the exemplary PCAS 106 shown in FIG. 3; however, it will be recognized that any suitable environment and system may be used and that any of the following blocks may be performed in any suitable order. For instance, process 500 may provide an example of the PCAS 106 performing block 404 in more detail.

At block 502, the at least one autonomous processor (e.g., the rules processor 306 and/or the predictive processor 308) generates a first result indicating whether the medical treatment for the patient is approved. At block 504, the user feedback processor 310 retrieves, from a decision repository and based on the medical procedure, questionnaire information indicating a plurality of questions associated with the patient or the medical treatment. At block 506, the user feedback processor 310 provides, to a user device 108, the questionnaire information indicating the plurality of questions. At block 508, the user feedback processor 310 receives user feedback associated with the questionnaire information. At block 510, the user feedback processor 310 generates a second result indicating whether the medical treatment for the patient is approved. At block 512, the stay processor 314 generates the authorization information indicating whether the medical treatment for the patient is approved based on the first result and the second result.

It will be appreciated that the figures of the present application and their corresponding descriptions are merely exemplary, and that the application is not limited to these exemplary situations.

It will further be appreciated by those of skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein as being performed by computing devices and/or components thereof may be carried out by according to processor-executable instructions and/or installed applications corresponding to software, firmware, and/or computer hardware.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the application and does not pose a limitation on the scope of the application unless otherwise claimed. No language in the specification should be construed as indicating any non-claimed element as essential to the practice of the application.

It will be appreciated that the examples of the application described herein are merely exemplary. Variations of these examples may become apparent to those of ordinary skill in the art upon reading the foregoing description. The inventors expect skilled artisans to employ such variations as appropriate, and the inventors intend for the application to be practiced otherwise than as specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

1. A method, comprising:
receiving, by a pre-certification authorization system (PCAS), treatment information indicating a medical treatment for a patient, wherein the treatment information is associated with a pre-certification request for the patient;
determining, by the PCAS, whether to provide pre-certification approval for the medical treatment for the patient based on using a plurality of approval processors to determine at least two results, wherein the plurality of approval processors comprises a user feedback processor configured to generate a first result of the at least two results, and at least one autonomous processor configured to generate one or more second results of the at least two results, wherein the user feedback processor generates the first result based on user feedback from a user device; and
providing, by the PCAS and based on the at least two results from the plurality of approval processors, authorization information indicating whether the pre-certification request for the patient is approved.

2. The method of claim 1, wherein the treatment information indicating the medical treatment for the patient comprises identification information associated with the patient, a medical condition associated with the patient, and a medical provider providing the medical treatment.

3. The method of claim 1, wherein the at least one autonomous processor comprises a rules processor and a predictive processor, and wherein determining whether to provide the pre-certification approval for the medical treatment for the patient is based on using a hierarchy indicating an order to use the rules processor, the predictive processor, and the user feedback processor.

4. The method of claim 3, wherein determining whether to provide the pre-certification approval for the medical treatment for the patient comprises:
determining, using the rules processor and based on the hierarchy, a third result based on applying one or more rules to the treatment information;
providing, based on the hierarchy, the third result to the user feedback processor;
in response to receiving the third result, providing, using the user feedback processor, questionnaire information associated with the treatment information to a user device; and
determining, using the user feedback processor, the first result based on user feedback from the user device.

5. The method of claim 4, wherein determining whether to provide the pre-certification approval for the medical treatment for the patient further comprises:
providing, based on the hierarchy, the first result to the predictive processor;
determining, using the predictive processor and based one or more machine learning models, a fourth result; and
providing the fourth result to a stay processor.

6. The method of claim 5, wherein determining whether to provide the pre-certification approval for the medical treatment for the patient further comprises:
based on the fourth result indicating approval of the pre-certification request for the patient, generating the authorization information; and
providing the authorization information indicating the approval of the pre-certification request to a health care treatment administration system.

7. The method of claim 4, wherein determining whether to provide the pre-certification approval for the medical treatment for the patient further comprises:
based on the first result indicating approval of the pre-certification request for the patient, providing the first result to a stay processor; and
providing the authorization information indicating the approval of the pre-certification request to a health care treatment administration system.

8. The method of claim 4, wherein determining whether to provide the pre-certification approval for the medical treatment for the patient further comprises:
retrieving the questionnaire information from a decision repository based on the medical treatment indicated by the treatment information.

9. The method of claim 1, further comprising:
determining an approval of the pre-certification request based on the at least two results;
in response to the approval, determining an approved treatment facility stay duration for the patient after the patient undergoes the medical treatment; and
generating the authorization information, wherein the authorization information indicates the approval of the pre-certification request and the approved treatment facility stay duration for the patient.

10. The method of claim 9, wherein determining the approved treatment facility stay duration for the patient after the patient undergoes the medical treatment comprises:
determining, using one or more parameters and the treatment information, an initial treatment facility stay duration, wherein each of the one or more parameters indicates a recommended stay duration associated with a particular type of medical treatment; and
inputting the initial treatment facility stay duration into one or more machine learning models to determine an extended treatment facility stay duration, wherein the approved treatment facility stay duration is the extended treatment facility stay duration determined by the one or more machine learning models.

11. A pre-certification authorization system (PCAS), comprising:
an intake system configured to receive treatment information indicating a medical treatment for a patient, wherein the treatment information is associated with a pre-certification request for the patient;
a user feedback processor configured to generate a first result based on user feedback from a user device;
at least one autonomous processor configured to generate one or more second results; and
a stay processor configured to:
determine whether to provide pre-certification approval for the medical treatment for the medical treatment for the patient based on the first result and the one or more second results; and
provide authorization information indicating whether the pre-certification request for the patient is approved.

12. The PCAS of claim 11, wherein the treatment information indicating the medical treatment for the patient comprises identification information associated with the patient, a medical condition associated with the patient, and a medical provider providing the medical treatment.

13. The PCAS of claim 11, wherein the at least one autonomous processor comprises a rules processor and a predictive processor.

14. The PCAS of claim 13, wherein the rules processor is configured to:
determine a third result based on applying one or more rules to the treatment information and a hierarchy indicating an order to use the rules processor, the predictive processor, and the user feedback processor; and
provide, based on the hierarchy, the third result to the user feedback processor; and
wherein the user feedback processor is configured to generate the first result based on the third result and the user feedback from the user device.

15. The PCAS of claim 14, wherein the user feedback processor is further configured to:
provide the first result to the predictive processor, and
wherein the predictive processor is configured to:
determine, based on one or more machine learning models, a fourth result; and
provide the fourth result to the stay processor.

16. The PCAS of claim 15, wherein the stay processor is configured to:
based on the fourth result indicating approval of the pre-certification request for the patient, generate the authorization information, and
wherein providing the authorization information comprises providing the authorization information indicating the approval of the pre-certification request to a health care treatment administration system.

17. The PCAS of claim 14, wherein the user feedback processor is further configured to:
based on the first result indicating approval of the pre-certification request for the patient, provide the first result to the stay processor, and
wherein the stay processor is configured to:
generate the authorization information based on receiving the first result, and
wherein providing the authorization information comprises providing the authorization information indicating the approval of the pre-certification request to a health care treatment administration system.

18. The PCAS of claim 11, wherein the stay processor is configured to:
in response to determining that the pre-certification request for the patient has been approved, determine an approved treatment facility stay duration for the patient after the patient undergoes the medical treatment; and
generate the authorization information, wherein the authorization information indicates the approval of the pre-certification request and the approved treatment facility stay duration for the patient.

19. The PCAS of claim 18, wherein the stay processor is configured to determine the approved treatment facility stay duration for the patient after the patient undergoes the medical treatment by:
determining, using one or more parameters and the treatment information, an initial treatment facility stay duration, wherein each of the one or more parameters indicates a recommended stay duration associated with a particular type of medical treatment;
inputting the initial treatment facility stay duration into one or more machine learning models to determine an extended treatment facility stay duration; and
determining the approved treatment facility stay duration as the extended treatment facility stay duration determined by the one or more machine learning models.

20. A non-transitory computer-readable medium having processor-executable instructions stored thereon, wherein the processor-executable instructions, when executed, facilitate:
receiving treatment information indicating a medical treatment for a patient, wherein the treatment information is associated with a pre-certification request for the patient;
determining whether to provide pre-certification approval for the medical treatment for the patient based on using a plurality of approval processors to determine at least two results, wherein the plurality of approval processors comprises a user feedback processor configured to generate a first result of the at least two results, and at least one autonomous processor configured to generate one or more second results of the at least two results, wherein the user feedback processor generates the first result based on user feedback from a user device; and
providing, based on the at least two results from the plurality of approval processors, authorization information indicating whether the pre-certification request for the patient is approved.
